(19) United States
(12) Patent Application Publication
(10) Pub. No.: US 2025/0155405 A1
(43) Pub. Date: May 15, 2025

(54) METHOD FOR IDENTIFYING UNSATURATED ORGANIC COMPOUND, AND MASS SPECTROMETRY SYSTEM

(71) Applicant: SHIMADZU CORPORATION, Kyoto-shi (JP)

(72) Inventors: Yuling Li, Shanghai (CN); Wenjian SUN, Shanghai (CN)

(21) Appl. No.: 18/930,566

(22) Filed: Oct. 29, 2024

(30) Foreign Application Priority Data
Nov. 9, 2023 (CN) 202311495122.8

(51) Int. Cl. G01N 27/623; G01N 1/44; G01N 33/92

(52) U.S. Cl. CPC G01N 27/623; G01N 1/44; G01N 33/92

(57) ABSTRACT

The present disclosure relates to the field of mass spectrometry, and specifically to a method for identifying an unsaturated organic compound and a mass spectrometry system, which are particularly suitable for implementing position identification of a carbon-carbon double bond of an unsaturated lipid. The identification method includes a derivatization reaction step of aziridinating a carbon-carbon double bond in the unsaturated organic compound by an aza-Prilezhaev reaction to obtain a derivatization product, and then specifically dissociating the derivatization product, and performing mass analysis. Compared with the existing method, the identification method has at least one of the advantages of mild reaction conditions, minimal over-derivatization, minimal side reactions, and high conversion rate.

### TECHNICAL FIELD

The present disclosure relates to the field of mass spectrometry, and specifically to a method for identifying an unsaturated organic compound and a mass spectrometry system.

### BACKGROUND ART

Lipids are important nutrients and components of biological cells, and are closely related to some important immune functions and metabolic defects. The lipid metabolites and pathways strategy (LIPID MAPS) has been initiated to establish a classification database to promote lipidomics study.

Complete lipid annotation and identification information include lipid type, fatty acyl compositionand position (sn-position), the number and positions of carbon-carbon double bonds, and geometry of the double bonds. In order to distinguish lipid isomers, in particular to identify the position of a double bond of an unsaturated lipid, many new mass spectrometry methods have been gradually developed. Current mainstream methods can be divided into three types. The first type is to use a separation technique such as liquid chromatography (Holc̆apek, M. et al. J. Chromatogr. A 2011, 1218, 5146) and ion mobility spectrometry (Mclean, J. A. et al. Anal. Chem. 2014, 86, 2107; Groessl, M. et al. Analyst 2015, 140, 6904; Fernandez-Lima, F. et al. Anal. Chem. 2019, 91, 5021; Ouyang, Z. et al. Nat. Commun. 2023, 14, 1535) to compare a peak position with that of a standard sample to identify the lipids, but this type of method requires a large number of standard samples. For lipidome analysis, there are more unknown samples, and a scope of application is limited. The second type is a method based on gas-phase ion activation, which can achieve selective fragmentation of a carbon-carbon double bond in an unknown lipid, and includes high-energy collision dissociation (Gross, M. L. et al. J. Am. Chem. Soc. 1983, 105, 5487), charge remote fragmentation (McLuckey, S. A. et al. Anal. Chem. 2019, 91, 9032), ultraviolet photodissociation (Brodbelt, J. S. et al. Anal. Chem. 2017, 89, 1516; J. Am. Chem. Soc. 2017, 139, 15681), ozone induced dissociation (Murphy, R. C. et al. Anal. Chem. 1996, 68, 3224; Blanksby, S. J. et al. J. Am. Chem. Soc. 2006, 128, 58; Anal. Chem. 2007, 79, 5013; Anal. Chem. 2008, 80, 303), electron impact excitation of ions from organic (Baba, T. et al. Anal. Chem. 2015, 87, 5837; Anal. Chem. 2017, 89, 7307), oxygen attachment dissociation (Takahashi, H. et al. Anal. Chem. 2018, 90, 7230), radical-directed dissociation (Blanksby, S. J. et al. Anal. Chem. 2012, 84, 7525), and the like.

However, in almost all dissociation methods based on gas-phase ion activation, unsaturated lipids are excessively fragmented, resulting in highly complex spectra, low abundance of relevant diagnostic ions at a position of a double bond, and poor sensitivity. Regarding analysis of a complex sample, a complex pretreatment or separation method is usually required. Due to a use of a sophisticated mass spectrometer and limited sensitivity, this type of method has not yet been applied to large-scale lipid analysis.

For identification of a low abundance lipid isomer in a complex biological sample, a third type of chemical derivatization-tandem mass spectrometry is currently the most commonly used method, and includes Paternò-Büchichi (P-B) reaction (Xia, Y. et al. Angew. Chem. Int. Ed. 2014, 53, 2592; Capriotti, A. L. et al. Anal. Chem. 2022, 94, 13117), epoxidation (Li, L. J. et al. Anal. Chem. 2019, 91, 1791; Hsu, C. C. et al. Anal. Chem. 2019, 91, 11905), singlet oxygen-ene reaction (Laskin, J. et al. Angew. Chem. Int. Ed. 2021, 60, 7559), aziridination (Guo, Y. L. et al. Anal. Chem. 2022, 94, 6216; Yan, X. et al. Angew. Chem. Int. Ed. 2022, 61, e202207098; Chen, S. M. et al. bioRxiv. doi: https://doi.org/10.1101/2022.04.24.489320; Chen, S. M. et al. Chinese Chem. Lett. 2024, 35, 108775), and other reactions (Brenna, J. T. et al. Anal. Chem. 2003, 75, 4925).

Xia and the others in “Deep-profiling of phospholipidome via rapid orthogonal separations and isomer-resolved mass spectrometry” (Nat. Comm., 2023, 14, 4263) integrate hydrophilic interaction liquid chromatography (HILIC), trapped ion mobility spectrometry (TIMS), and isomer-resolved tandem mass spectrometry (MS/MS), and develop a phospholipidome deep analysis system having automatic data analysis capabilities to achieve rapid, sensitive, and high-coverage qualitative and quantitative analysis of phospholipidomes in various biological samples. In this paper, off-line Paternò-Büchi (P-B) reaction is used to label a carbon-carbon double bond of the phospholipidome.

Li lab (Li, L. J. et al. Anal. Chem. 2019, 91, 1791) and Hsu lab (Hsu, C. C. et al. Anal. Chem. 2019, 91, 11905) have proposed a method for identifying a position of a carbon-carbon double bond of an unsaturated lipid by the epoxidation reaction. Based on the Prilezhaev mechanism, mCPBA is used to react with the double bond to convert the double bond into oxirane. This three-membered ring structure is easily fragmented in the subsequent CID, thereby producing diagnostic ions that can indicate the position of the double bond.

However, the epoxidation does not introduce a functional group that is easily ionized, and at the same time, due to a problem of reaction yield, sensitivity of detection is low, and thus a great difficulty is still present in analysis of low-content lipids. Regarding a lipid containing a plurality of double bonds, a variety of over-oxidation byproducts is formed by the reaction, which not only greatly reduces the sensitivity, but also greatly increases a difficulty of analysis.

Aziridination is a good strategy for introducing an easily ionized site. Yan and the others disclose a new mass tag in a patent of WO2022/216767A1 in which a combination of HOSA reagent, pyridine, and ethyl trifluoropyruvate is used to convert double bond to aziridine. In the reaction, electron-deficient ketone is used as a catalyst to first react with the nitrogen source HOSA to produce oxaziridine which is a key intermediate. The intermediate then reacts with a double-bond compound to produce aziridine.

Guo and the others disclose a method for identifying a position of a double bond using a chloramine reagent in a patent of CN114166921A. In this patent, a sodium salt of N-chloro-4-methylbenzenesulfonamide is mainly used as a reaction reagent for labeling a carbon-carbon double bond.

However, the methods for labeling a carbon-carbon double bond of a lipid in the related art have problems.

### SUMMARY OF THE INVENTION

According to a continuous and extensive study on the related art, the inventors have found that the method for labeling a carbon-carbon double bond of a lipid in the related art has at least one problem, such as harsh reaction conditions, serious over-derivatization, many side reactions, and low conversion rate.

A first aspect of the present application relates to a method for identifying an unsaturated organic compound, the method including:

a derivatization reaction step of aziridination of a carbon-carbon double bond in the unsaturated organic compound by an aza-Prilezhaev reaction to obtain a derivatization product, wherein a derivatization reagent is used in the aza-Prilezhaev reaction containing a compound represented by the following general formula (1):

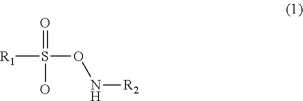

in the general formula (1),

R₁ is selected from a substituted or unsubstituted aromatic group having 6 to 18 ring-forming carbon atoms, and

R₂ is selected from H, a substituted or unsubstituted linear alkyl, alkoxy, or thioalkyl group having 1 to 20 carbon atoms, a substituted or unsubstituted branched or cyclic alkyl, alkoxy, or thioalkyl group having 3 to 20 carbon atoms, a substituted or unsubstituted alkenyl or alkynyl group having 2 to 20 carbon atoms, or a substituted or unsubstituted aromatic or heteroaromatic ring system having 5 to 30 aromatic ring atoms;

a dissociation step of dissociating the ionized derivatization product such that the ionized derivatization product is cleaved at a site corresponding to an original site having the carbon-carbon double bond to obtain a plurality of daughter ions; and

a mass analysis step of measuring mass numbers of the plurality of daughter ions to determine a position of the carbon-carbon double bond in the unsaturated organic compound.

Optionally, R₂ has a mass number greater than 80 Da.

Optionally, the compound represented by the general formula (1) is as follows:

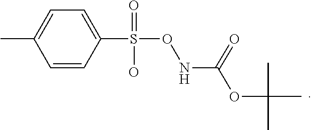

Optionally, a solvent of the derivatization reagent is an acidic solvent.

Optionally, the acidic solvent is one of trifluoroethanol, hexafluoroisopropanol, and perfluoro-tert-butanol, or a combination thereof.

Optionally, a reaction temperature of the aza-Prilezhaev reaction is 20° C. to 100° C.

Optionally, the unsaturated organic compound is an unsaturated lipid.

Optionally, the unsaturated lipid is fatty acyl, glycerolipid, glycerophospholipid, sphingolipid, a sterol lipid, a prenol lipid, a saccharolipid, or polyketide.

A second aspect of the present application relates to a mass spectrometry system including: a derivatization reactor; an ion source; a dissociation device; and a mass spectrometer. The derivatization reactor mixes and reacts a sample with an aza-prilezhaev derivatization reagent, and a carbon-carbon double bond in an unsaturated organic compound in the sample is aziridinated by an aza-Prilezhaev reaction to obtain a derivatization product, the derivatization reagent contains a compound represented by the following general formula (1):

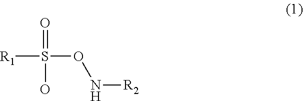

in the general formula (1),

R₁ is selected from a substituted or unsubstituted aromatic group having 6 to 18 ring carbon atoms, and

R₂ is selected from H, a substituted or unsubstituted linear alkyl, alkoxy, or thioalkyl group having 1 to 20 carbon atoms, a substituted or unsubstituted branched or cyclic alkyl, alkoxy, or thioalkyl group having 3 to 20 carbon atoms, a substituted or unsubstituted alkenyl or alkynyl group having 2 to 20 carbon atoms, or a substituted or unsubstituted aromatic or heteroaromatic ring system having 5 to 30 aromatic ring atoms;

The ion source receives and ionizes the derivatization product; the dissociation device dissociates the derivatization product ionized by the ion source such that the derivatization product is fragmented at a site corresponding to an original site having the carbon-carbon double bond to obtain a plurality of daughter ions; and the mass spectrometer measures mass numbers of the daughter ions to determine a position of the carbon-carbon double bond in the unsaturated organic compound.

Optionally, R₂ has a mass number greater than 80 Da.

Optionally, the compound represented by the general formula (1) is as follows:

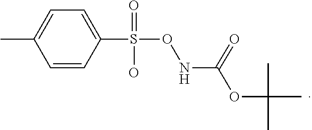

Optionally, the ion source is an electrospray ionization source, a nanoelectrospray ionization source, a desorption electrospray ionization source, an atmospheric pressure chemical ionization source, an atmospheric pressure photoionization source, or a matrix-assisted laser desorption ionization source.

Optionally, the dissociation device is one or more of a high-energy collision dissociation device, a collision induced dissociation device, an oxygen attachment dissociation device, a hydrogen attachment dissociation device, an electron capture dissociation device, a radical directed dissociation device, an ultraviolet photodissociation device, and a charge-remote site fragmentation device.

Optionally, the dissociation device is the collision induced dissociation device, and dissociation energy of the collision induced dissociation device is 30 eV to 40 eV.

Optionally, the derivatization reactor is an off-line reaction device and includes a reaction vessel in which the sample and the derivatization reagent are mixed, and an acceleration control unit configured to accelerate collision of molecules in the reaction vessel.

Optionally, the derivatization reactor is an on-line reaction device and includes a communication device and an acceleration control unit. A first liquid inlet is disposed in the communication device and communicates with a liquid inlet pipeline for the sample. A second liquid inlet is disposed in the communication device and communicates with a liquid inlet pipeline for the derivatization reagent. A product outlet is disposed in the communication device, and the derivatization product is conveyed through the product outlet to the ion source. The acceleration control unit accelerates collision of molecules in the communication device.

Optionally, the acceleration control unit is one or more of a temperature control unit, an ultrasonic device, a microwave device, an infrared device, and an oscillation device.

Optionally, the temperature control unit controls a reaction temperature of the aza-Prilezhaev reaction at 20° C. to 100° C.

Optionally, the mass spectrometry system further includes a liquid chromatography device disposed in the liquid inlet pipeline for the sample.

Optionally, the mass spectrometry system further includes a mass filter disposed between the ion source and the dissociation device.

Optionally, the mass spectrometry system further includes an ion mobility spectrometer disposed between the ion source and the dissociation device.

Compared with the existing identification methods, the method for identifying an unsaturated organic compound according to the present application has at least one of the following advantages.

a. Many types of identifiable information: for example, a position of a carbon-carbon double bond in a fatty acyl chain, an sn-position of the fatty acyl chain, and a cis-trans isomer orientation of the carbon-carbon double bond can be determined.
b. Good substrate versatility: applicable to various substrates such as FA, GP, ST, SP, and GL.
c. Mild reaction condition: metal catalysts and additional additives are not required, UV, electrolysis, high temperature, inert atmosphere, and dry environment are not required, and there is no strong redox agent. The reaction can be completed quickly at room temperature or slightly increased temperature, and a reagent and a product have good stability.
d. High conversion rate: the conversion rate can reach more than 90%.
e. Few side reactions and enhanced ionization efficiency.
f. Minimal over-derivatization: usually only a single carbon-carbon double bond in each fatty acyl chain of an unsaturated organic compound is labeled, or by optimizing reaction conditions (such as temperature and time), only a single carbon-carbon double bond in most molecules is labeled. A spectrum is simple and easy to analyze.
g. Run on-line.

### DESCRIPTIONS OF THE DRAWINGS

FIG. 1 is a schematic flowchart of a method for identifying a position and a cis-trans isomer orientation of a carbon-carbon double bond of an unsaturated lipid according to a first embodiment of the present disclosure.

FIG. 2 is a yield result obtained by an aza-Prilezhaev reaction at different temperatures for 10 minutes in the first embodiment of the present disclosure.

FIG. 3 is a graph showing a change in a conversion rate reflected by mass spectral peak intensity of a target substance over time at a reaction temperature of 50° C. in the first embodiment of the present disclosure.

FIG. 4 is a system diagram of a mass spectrometry system for off-line analysis of a derivatization product according to the first embodiment of the present disclosure.

FIG. 5 is a graph showing a change in mass spectral peak intensity of an aziridinated lipid at different times according to the first embodiment of the present disclosure.

FIG. 6 is a graph showing a fold change in mass spectral peak intensity before and after a derivatization reaction according to the first embodiment of the present disclosure.

FIG. 7 is a diagram showing a reaction flow of the identification method according to the first embodiment of the present disclosure.

FIG. 8 is a mass spectrum of a C18:1 (9Z) standard sample after aziridination according to the first embodiment of the present disclosure.

FIG. 9 is a mass spectrum of a C18:1 (6Z) standard sample after aziridination according to the first embodiment of the present disclosure.

FIG. 10 is a mass spectrum of a PC16:0/18:1 (9Z) standard sample after aziridination according to the first embodiment of the present disclosure.

FIG. 11 is a mass spectrum of a PC18:1 (9Z)/16:0 standard sample after aziridination according to the first embodiment of the present disclosure.

FIG. 12 is a schematic diagram of a molecular fragmentation process of PC16: 0/18:1(9Z) after aziridination according to the first embodiment of the present disclosure.

FIG. 13 is a schematic diagram of a molecular fragmentation process of PC18:1 (9Z)/16:0 after aziridination according to the first embodiment of the present disclosure.

FIG. 14 is an ion mobility spectrometry spectrum obtained from aziridine-C18:1 (9E) [M+2Na-H]⁺, C18:1 (9Z) [M+2Na-H]⁺, and a mixture thereof according to the first embodiment of the present disclosure.

FIG. 15 is a mass spectrum obtained from aziridine-C20:4 (5Z, 8Z, 11Z, 14Z) according to the first embodiment of the present disclosure.

FIG. 16 is a tandem mass spectrum obtained from aziridineC20:4 (5Z, 8Z, 11Z, 14Z) according to the first embodiment of the present disclosure.

FIG. 17 is a system diagram of a mass spectrometry system for on-line analysis of a derivatization product according to a second embodiment of the present disclosure.

LIST OF REFERENCE NUMERALS

ion source 1, ion mobility spectrometer 2, mass filter 3, dissociation device 4, mass analyzer 5, ion guide device 6, derivatization reactor 7, communication device 71, first liquid inlet 711, second liquid inlet 712, product outlet 713, acceleration control unit 72, 81 liquid inlet pipeline for sample, liquid inlet pipeline for derivatization reagent 82, chromatography column 9.

### DETAILED DESCRIPTION

Technical schemes in embodiments of the present disclosure will be clearly and completely described below with reference to the accompanying drawings in the embodiments of the present disclosure, and obviously, the described embodiments are merely a part of the embodiments of the present disclosure, and are not all embodiments. All other embodiments obtained by those skilled in the art without creative efforts shall fall within the scope of the present disclosure.

### Lipids

An unsaturated organic compound according to an embodiment of the present disclosure at least includes unsaturated lipids, such as unsaturated fatty acyl, glycerolipid, glycerophospholipid, sphingolipid, a sterol lipid, a prenol lipid, a saccharolipid, or polyketide, and is in particular suitable for fatty acyl/fatty acid, glycerophospholipid, the sterol lipid, the sphingolipid, and the glycerolipid.

Rule for naming lipids: lipids in the following descriptions use the A-nomenclature, and for example, in FA 18:1 (9Z), FA represents fatty acid, 18 represents the number of carbon atoms, 1 represents the number of carbon-carbon double bonds, 9 represents a position of the carbon-carbon double bonds, and Z represents that a geometric configuration is cis.

In TG18:1 (9Z)/18:1 (9Z)/18:1 (9Z), three fragments connected by “/” respectively represent lipid structures respectively corresponding to three fatty acyl chains in triglyceride.

In addition, English abbreviations below represent the following lipid types.
CE: cholesterol ester
PC: phosphatidylcholine
FA: fatty acyl/fatty acid
GP: glycerophospholipid
ST: sterol lipid
SP: sphingolipid
GL: glyceride
TG: triglyceride

### First Embodiment

### <Overall Flow>

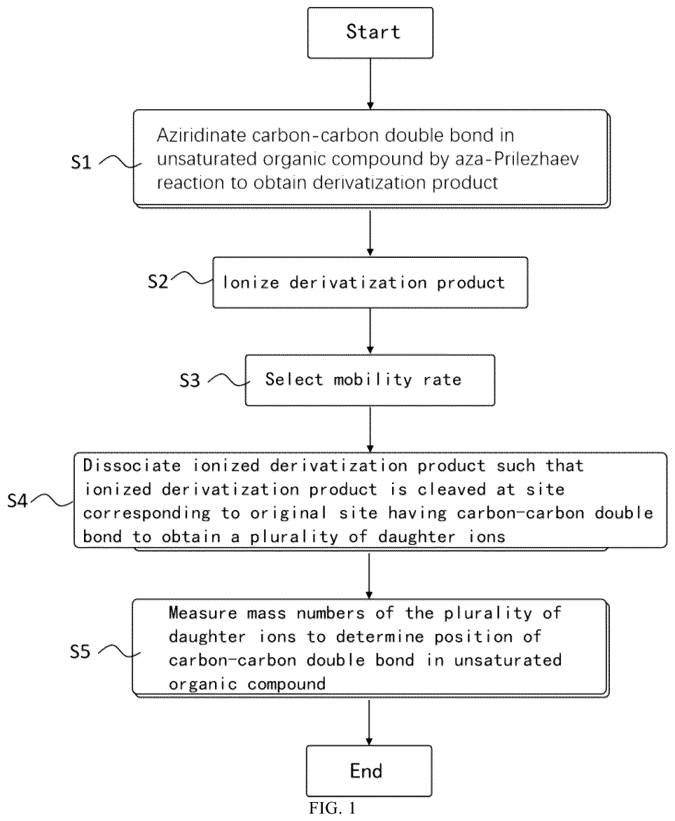

FIG. 1 is a schematic flowchart of a method for identifying a position (including a position in a fatty acyl chain and sn-position) and a cis-trans isomer orientation of a carbon-carbon double bond of an unsaturated lipid according to the present embodiment.

Referring to FIG. 1, the identification method includes the following steps.

A derivatization reaction step S1 is to aziridinate a carbon-carbon double bond in an unsaturated organic compound by an aza-Prilezhaev reaction to obtain a derivatization product.

An ionization step S2 is to ionize the derivatization product.

A mobility rate selection step S3 is to select, according to an ion mobility rate, the derivatization product obtained by aziridinating the carbon-carbon double bond.

A dissociation step S4 is to dissociate the ionized derivatization product such that the ionized derivatization product is cleaved at a site corresponding to an original site having the carbon-carbon double bond to obtain a plurality of daughter ions.

A mass analysis step S5 is to measure mass numbers of the plurality of daughter ions to determine a position of the carbon-carbon double bond in the unsaturated organic compound.

In the above steps, before the derivatization reaction step S1, liquid chromatography or gas chromatography and other technologies can also be used to separate the unsaturated organic compound, and then to separate components with the carbon-carbon double bond to reduce complexity of a spectrum and improve an analysis rate.

In the mass analysis step S5, the measured daughter ions at least include diagnostic ions, that is, daughter ions generated by being cleaved at the site of the carbon-carbon double bond, or daughter ions generated by being cleaved at a sn-position.

In the present embodiment, the ionization step S2 occurs after the derivatization reaction step S1, the mobility rate selection step S3 occurs after the ionization step S2, the dissociation step S4 occurs after the mobility rate selection step S3, and the mass analysis step S5 occurs after the dissociation step S4. In other embodiments of the present disclosure, the order of some steps can also be interchanged, and the present disclosure does not limit this.

### <Aza-Prilezhaev Reaction>

In the derivatization reaction step S1, the carbon-carbon double bond is labeled by the aza-Prilezhaev reaction. A reaction mechanism of the aza-Prilezhaev reaction is as follows.

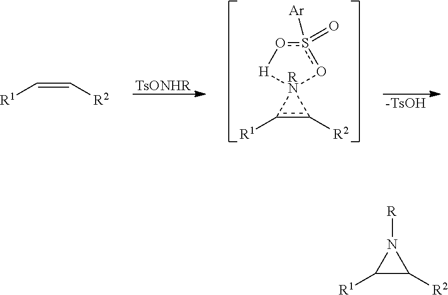

Unlike a two-step catalytic reaction of O-Sulfohydroxylamine (HOSA), pyridine, and double bonds, the aza-Prilezhaev reaction does not require a catalyst, and O-arylsulfonylhydroxylamine and the double bonds undergo only one step of a concerted reaction to remove a molecule of aromatic sulfonic acid and obtain N—R aziridine.

The derivatization reaction step S1 can be completed off-line, that is, an experimenter completes the steps of mixing raw materials, controlling the temperature, and the like by himself or herself, and the derivatization reaction step S1 can also be completed on-line, that is, analysis equipment completes the step according to a pre-set program. In present embodiment, the derivatization reaction step S1 is described as being completed off-line. In a second embodiment, a mass spectrometry system that can complete the derivatization reaction step S1 on-line is also provided.

### a) Aza-Prilezhaev Reagent

### a. 1 Mass Label

A derivatization reagent includes a mass label dissolved in an acidic solvent, and the mass label is a compound represented by the following general formula (1).

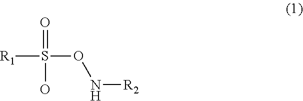

In the general formula (1),

R₁ is selected from a substituted or unsubstituted aromatic group having 6 to 18 ring-forming carbon atoms, and

R₂ is selected from H, a substituted or unsubstituted linear alkyl, alkoxy, or thioalkyl group having 1 to 20 carbon atoms, a substituted or unsubstituted branched or cyclic alkyl, alkoxy, or thioalkyl group having 3 to 20 carbon atoms, a substituted or unsubstituted alkenyl or alkynyl group having 2 to 20 carbon atoms, or a substituted or unsubstituted aromatic or heteroaromatic ring system having 5 to 30 aromatic ring atoms.

R₁ uses an aromatic group to improve stability of the reagent. R₂ is usually a group having a mass number greater than 15 Da. Exemplarily, R₂ is a group having a mass number greater than 50 Da. More exemplarily, R₂ is a group having a mass number greater than 80 Da, or R₂ is a group having a mass number greater than 100 Da. Reasonable increase in the mass number of R₂ can effectively prevent overlap of mass peaks in the spectrum. In some embodiments, R₂ may contain a group having a large steric hindrance such as a methyl group or a benzene ring, and examples thereof include tert-butyloxycarbonyl or benzoyl.

In the present embodiment, the mass label in the aza-Prilezhaev reagent is N-Boc-O-tosylhydroxylamine (CAS: 105838-14-0)

Other optional mass labels of the aza-Prilezhaev reagent that are readily available and CAS numbers of these mass labels are listed in the following table.

| Mass label of aza-Prilezhaev reagent | CAS number |
|---|---|
| O-m-Trifluormethylphenylsulfonyl-p- | 80953-44-2 |
| methylbenzylhydroxylamin | |
| O-m-Trifluormethylphenylsulfonyl-m- | 80953-46-4 |
| trifluormethylbenzylhydroxylamin | |
| N-benzyl-O-(m- | 80953-41-9 |
| (trifluoromethyl)benzenesulfonyl)hydroxylamine | |
| O-Phenylsulfonyl-m- | 114467-10-6 |
| trifluormethylbenzylhydroxylamin | |
| O-p-Bromphenylsulfonyl-m- | 114467-08-2 |
| trifluormethylbenzylhydroxylamin | |
| O-p- | 1219619-46-1 |
| trifluoromethylbenzenesulfonylhydroxylamine | |
| O-Mesitylenesulfonylhydroxylamine | 36016-40-7 |
| O-p-Toluenesulfonylhydroxylamine | 52913-14-1 |
| O-(mesitylsulfony1)-N-methylhydroxylamine | 65962-98-3 |
| N-phenyl-N-(tosyloxy)benzamide | 83125-89-7 |
| N-methyl-O-tosylhydroxylamine | 25370-97-2 |
| tert-butyl nosyloxycarbamate | 164078-85-7 |
| tert-butyl ((methylsulfonyl)oxy)carbamate | 219844-41-4 |
| N-(tert-butoxycarbonyl)-O- | 36016-39-4 |
| (mesitylsulfonyl)hydroxylamine | |

### a.2 Solvent

The acidic solvent used for the derivatization reagent may be an organic acid such as trifluoroethanol, hexafluoroisopropanol, and perfluoro-tert-butanol, and may be an inorganic acid. Hexafluoroisopropanol is exemplary.

### b) Reaction Temperature

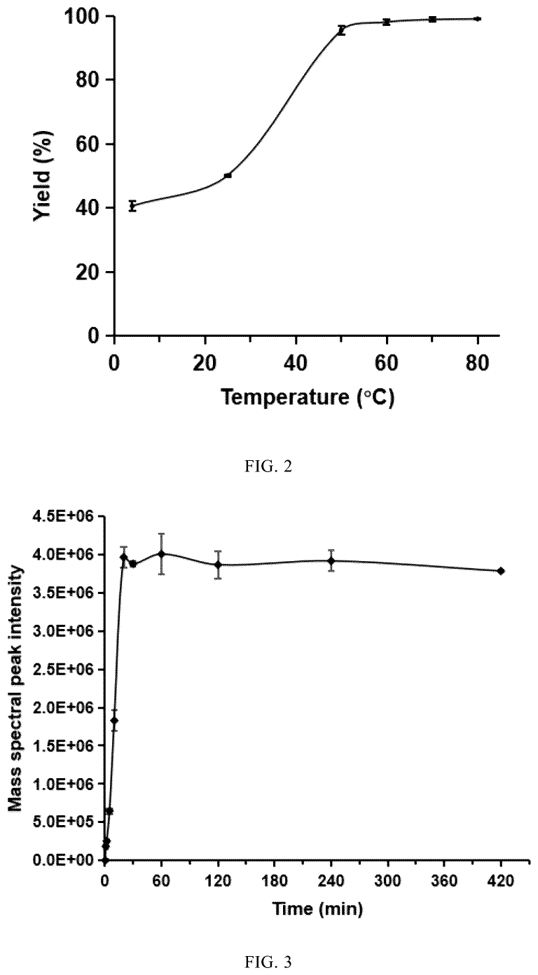

In an example of a reaction for 10 minutes, N-Boc-O-tosylhydroxylamine was made as a mass label and hexafluoroisopropanol was made as a solvent. FIG. 2 shows a reaction yield result at different temperatures for 10 minutes. According to the experiment, the yield of aza-Prilezhaev could be greatly improved as the temperature increased from about 20° C., and tended to be stable at about 50° C. to 55° C. Exemplarily, the reaction temperature was set in a range of 20° C. to 55° C., more exemplarily in a range of 30° C. to 55° C., and for example, 50° C. A higher yield could be obtained within this temperature range, while reducing occurrence of side reactions.

### c) Reaction Time

FIG. 3 is a graph showing a change in a conversion rate reflected by mass spectral peak intensity of a target substance over time at a reaction temperature of 50° C. Referring to FIG. 3, it was found in the study that at the reaction temperature of 50° C., the aza-Prilezhaev reaction rate was very high. The peak intensity could be detected in 30 seconds. If the reaction exceeded 10 minutes, the conversion rate could be close to 100%.

For further details and mechanism discussion of the aza-Prilezhaev reaction, “Stereospecific Alkene Aziridination Using a Bifunctional Amino-Reagent: An Aza-Prilezhaev Reaction” (J. Am. Chem. Soc. 2018, 140, 17846) published by John F. Bower can be referred to, and descriptions are not made here.

### <IMS-MS/MS>

### a) Configuration of System

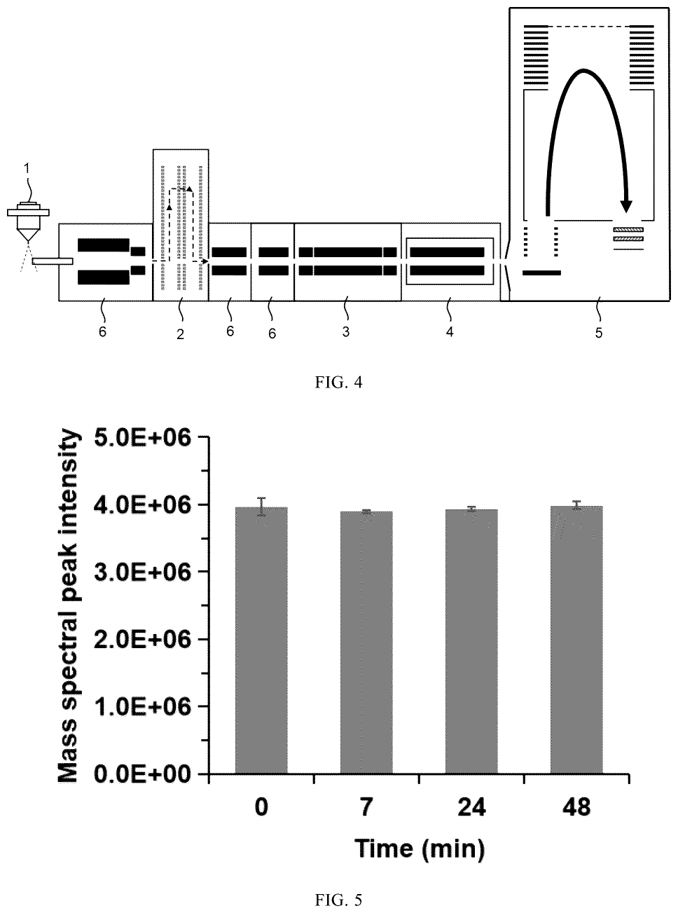

Referring to FIG. 4, a system for off-line analysis of a derivatization product includes an ion source 1, an ion mobility spectrometer 2, and a tandem mass spectrometer connected in series.

### b) Ion Source 1

The ionization step S2 is completed by the ion source 1.

The ion source 1 includes an ion source selected from the group consisting of an electrospray ionization source (ESI); an atmospheric pressure photoionization source (APPI); an atmospheric pressure chemical ionization source (APCI); a matrix-assisted laser desorption ionization source (MALDI); a laser desorption ionization source (LDI); an atmospheric pressure ionization source (API); a desorption ionization on silicon source (DIOS); an electron impact ion source (EI); a chemical ionization source (CI); a field ionization source (FI); a field desorption ion source (FD); an inductively coupled plasma ion source (ICP); a fast atom bombardment ion source (FAB); a liquid secondary ion mass spectrometry ion source (LSIMS); an electrospray desorption ionization source (DESI); a nickel-63 radioactive ion source; an atmospheric pressure matrix-assisted laser desorption ionization ion source; a thermal spray ion source; an atmospheric sampling glow discharge ionization source (ASGDI); a glow discharge ion source (GD); an impactor ion source; a direct analysis in real time ion source (DART); a laser spray ionization source (LSI); a sonic spray ionization source (SSI); a matrix-assisted inlet ionization source (MAII); a solvent assisted inlet ionization source (SAII); a Penning ionization source; a laser ablation electrospray ionization source (LAESI); and a He plasma ionization source (HePI). Exemplarily, the ion source 1 is the electrospray ionization source, a nanoelectrospray ionization source, a desorption electrospray ionization source, the atmospheric pressure chemical ionization source, the atmospheric pressure photoionization source, or the matrix-assisted laser desorption ionization source. In the present embodiment, the ion source is exemplarily the electrospray ionization source.

### c) Ion Mobility Spectrometer 2

The mobility rate selection step S3 is completed by the ion mobility spectrometer 2. One or more ion guide devices 6 may be disposed between the ion mobility spectrometer 2 and the ion source.

The ion mobility spectrometer 2 includes an ion mobility rate analysis device selected from the group consisting of a drift tube ion mobility spectrometer (DTIMS), a differential mobility analysis (DMA) device, a field asymmetric-waveform ion-mobility spectrometry (FAIMS) device, a travelling wave ion mobility spectrometry (TW-IMS), a differential mobility spectrometry (DMS) device, a transverse modulation ion mobility spectrometry, a trapped ion mobility spectrometry (TIMS), and a U-shaped ion mobility analyzer (UMA).

In the present embodiment, the ion mobility spectrometer 2 is exemplarily the U-shaped ion mobility analyzer. A structure of the U-shaped ion mobility analyzer and an introduction of a filtering mode suitable for the identification method can refer to Chinese patent of CN113495112A, and descriptions are not made here. Similarly, the identification method according to the embodiment of the present disclosure does not limit the type of the ion mobility spectrometer used.

The ion mobility spectrometer 2 can bring a second dimension of data to tandem mass spectrometry analysis. Based on a difference in ion mobility rates, isomers can be distinguished. In particular, after the aza-Prilezhaev reaction aziridinates the carbon-carbon double bond, a difference in molecular structures is enhanced. In some embodiments, a difference in the position of the carbon-carbon double bond or the cis-trans isomer orientation can be identified using an ion mobility spectrogram.

### d) Tandem Mass Spectrometer

The tandem mass spectrometer includes a mass filter 3, a dissociation device 4, and a mass analyzer 5 connected in series. One or more ion guide devices 6 may also be provided between the ion mobility spectrometer 2 and the mass filter 3.

The tandem mass spectrometer may include one or more mass filters 3 selected from the group consisting of a quadrupole mass filter; a 2D or linear quadrupole ion trap; a Paul or 3D quadrupole ion trap; a Penning ion trap; an ion trap; a magnetic sector mass filter; a time-of-flight mass filter; and a Wien filter.

The dissociation step S4 is completed by the dissociation device 4. The tandem mass spectrometer may include one or more dissociation devices 4 selected from the group consisting of a collision induced dissociation (CID) device; a surface induced dissociation (SID) device; an electron transfer dissociation (ETD) device; an electron capture dissociation (ECD) device; an electron collision or impact dissociation device; a photo induced dissociation (PID) device; a laser induced dissociation device; an infrared radiation induced dissociation device; an ultraviolet radiation induced dissociation device; a nozzle-separator interface dissociation device; an in-source dissociation device; an in-source collision induced dissociation device; a heat or temperature source dissociation device; an electric field induced dissociation device; a magnetic field induced dissociation device; an ion-ion reaction dissociation device; an ion-molecule reaction dissociation device; an ion-atom reaction dissociation device; an ion-metastable ion reaction dissociation device; an ion-metastable molecule reaction dissociation device; and an electron ionization dissociation (EID) device. Exemplarily, dissociation energy of the collision induced dissociation device 4 is 30 eV to 40 eV. By reasonably setting the dissociation energy, a dissociation process can be site-specific, that is, a site where an aziridine ring is located is highly selectively cleaved to avoid generation of side reactions.

In the present embodiment, in the dissociation step S4, not only a site of the carbon-carbon double bond of the derivatization product can be cleaved, but also sn-positions of some derivatization products can be cleaved to detect the sn-position of the carbon-carbon double bond. The dissociation step S4 can be completed by applying a single dissociation or by a plurality of dissociations, and the present application does not limit this.

The mass analysis step S5 is completed by the mass analyzer 5, or by the mass filter 3 and the mass analyzer 5. The tandem mass spectrometer may include the mass analyzer 5 selected from the group consisting of a quadrupole mass analyzer; a 2D or linear quadrupole mass analyzer; a Paul or 3D quadrupole mass analyzer; a Penning trap mass analyzer; an ion trap mass analyzer; a magnetic sector mass analyzer; an ion cyclotron resonance (ICR) mass analyzer; a fourier transform ion cyclotron resonance (FTICR) mass analyzer; an electrostatic mass analyzer configured to generate an electrostatic field having a quadrupole logarithmic potential distribution; a fourier transform electrostatic mass analyzer; a fourier transform mass analyzer; a time-of-flight mass analyzer; an orthogonal acceleration time-of-flight mass analyzer; and a linear acceleration time-of-flight mass analyzer.

In the present embodiment, the tandem mass spectrometer is a Q-TOF tandem mass spectrometer, that is, the mass filter 3 is a quadrupole mass filter, and the mass analyzer 5 is a time-of-flight mass analyzer. The dissociation device 4 is a collision induced dissociation device.

### <Experimental Results>

### a) Stability

In addition, the aziridinated lipid obtained by the reaction can exist stably. FIG. 5 is a graph showing a change in detection peak intensity of the aziridinated lipid at different times according to the first embodiment of the present disclosure. Referring to FIG. 5, after the aziridinated lipid was placed for more than 48 hours, the detection peak intensity was substantially unaffected, and needs for different types of tests could be satisfied.

### b) Sensitivity

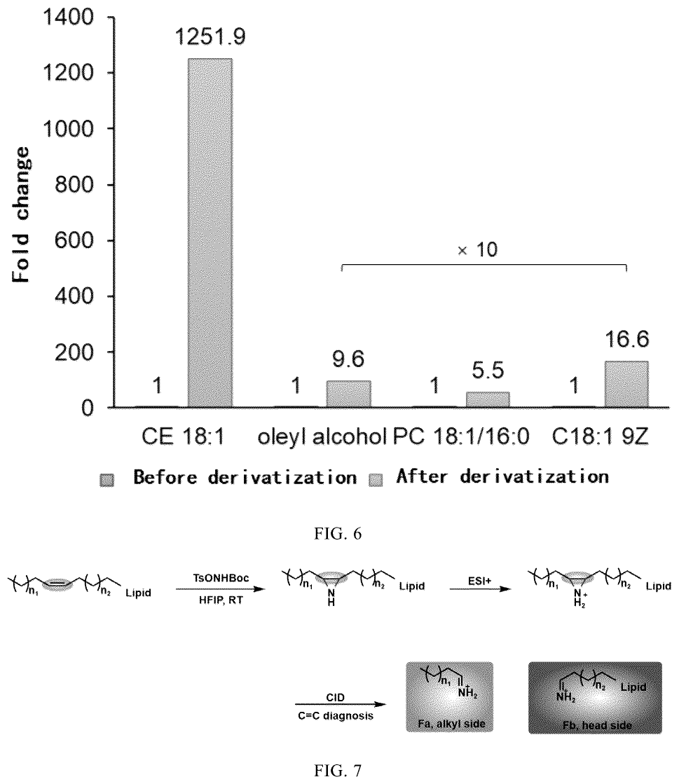

FIG. 6 is a graph showing a fold change in mass spectral peak intensity before and after the derivatization reaction according to the first embodiment of the present disclosure. Referring to FIG. 6, due to introduction of a group that could be easily ionized, the aza-Prilezhaev reaction could also improve sensitivity of mass spectrometry detection by performing aziridinization on the carbon-carbon double bond. Specifically, mass spectrometry signal intensity of a parent ion can be increased by 1 to 3 orders of magnitude.

### c) Distinguishing Isomers Having Carbon-Carbon Double Bond at Different Positions

### c.1. Identification of Position of Carbon-Carbon Double Bond in Fatty Chain

FIG. 7 is a diagram showing a reaction flow of the identification method according to the first embodiment of the present disclosure. Referring to FIG. 7, when the aziridine ring was specifically cleaved, two different situations may occur, that is, —NH₂⁺—was attributed to the fatty chain or to a head group. Two groups having different mass-to-charge ratios were formed accordingly, and thus the position of the carbon-carbon double bond could be determined based on these two mass peaks.

C18:1 (9Z) and C18:1 (6Z) were isomers, and a difference was in different positions of the carbon-carbon double bonds on the fatty chains. Specifically, the carbon-carbon double bond of C18:1 (9Z) was located between the ninth and tenth carbon atoms of the main chain, whereas the carbon-carbon double bond of C18:1 (6Z) was located between the sixth and seventh carbon atoms of the main chain. The following describes how to determine the position of the carbon-carbon double bond based on the mass spectrometry spectrum.

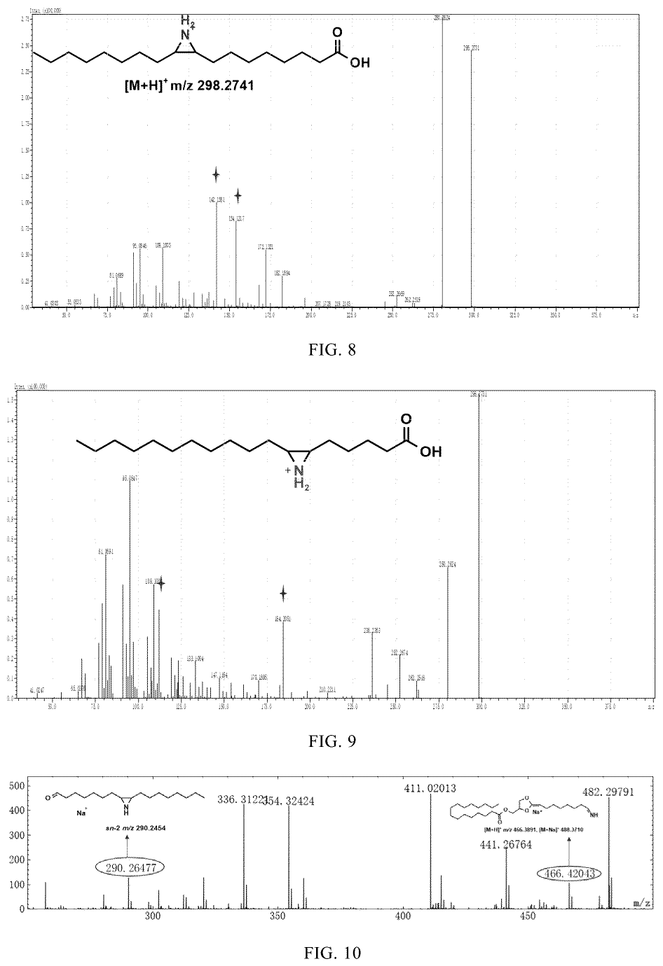

FIG. 8 is a mass spectrum of the C18:1 (9Z) standard sample after aziridination. Referring to a molecular structure after aziridination shown in FIG. 8, when the aziridine ring was specifically cleaved by the dissociation device 4, the fatty chain side was cleaved to form C₈H₁₇—CH═NH₂⁺daughter ions, that is, a mass peak of m/z=142, and the carboxylic acid group side was cleaved to form C₈H₁₃O—CH═NH₂⁺daughter ions, that is, a mass peak of m/z=154.

FIG. 9 is a mass spectrum of the C18:1 (6Z) standard sample after aziridination. Referring to a molecular structure after aziridination shown in FIG. 9, when the aziridine ring was specifically cleaved by the dissociation device 4, the fatty chain side was cleaved to form C₁₁H₂₃—CH═NH₂⁺daughter ions, that is, a mass peak of m/z=184, and the carboxylic acid group side was cleaved to form C₅H_{y}O—CH═NH₂⁺daughter ions, that is, a mass peak of m/z=112.

According to the above, since mass numbers of the daughter ions on the fatty acyl chain side and the daughter ions on the lipid side, which were obtained by cleaving the carbon-carbon double bond at different positions, were different, the specific position of the carbon-carbon double bond in the fatty acyl chain could be determined based on whether (peak intensity or) the above characteristic mass peaks and characteristic mass peaks corresponding to other different positions were present. Since the site of the aziridine ring was easily cleaved specifically after applying the dissociation energy, the peak intensity of the above characteristic mass peaks was strong, and sensitivity and accuracy of the detection were high.

c.2. Identification of sn-Position of Carbon-Carbon Double Bond

FIG. 10 is a mass spectrum of a PC 16:0/18:1 (9Z) standard sample after aziridination.

FIG. 11 is a mass spectrum of a PC 18:1 (9Z)/16:0 standard sample after aziridination.

Referring to “Large-scale lipid analysis with C═C location and sn-position isomer resolving power” (Nat. Commun., 2020, 11, 375) published by Cao and others, a difference in sn-positions of PC16:0/18:1 (9Z) and PC18:1 (9Z)/16:0 could be distinguished by the characteristic peaks of m/z=380, 396, 466. The above characteristic peaks corresponded to a PB reaction. Correspondingly, corresponding to the aza-Prilezhaev aziridination reaction, the difference in the sn-positions could be identified by the characteristic peaks of m/z=274, 290, 360.

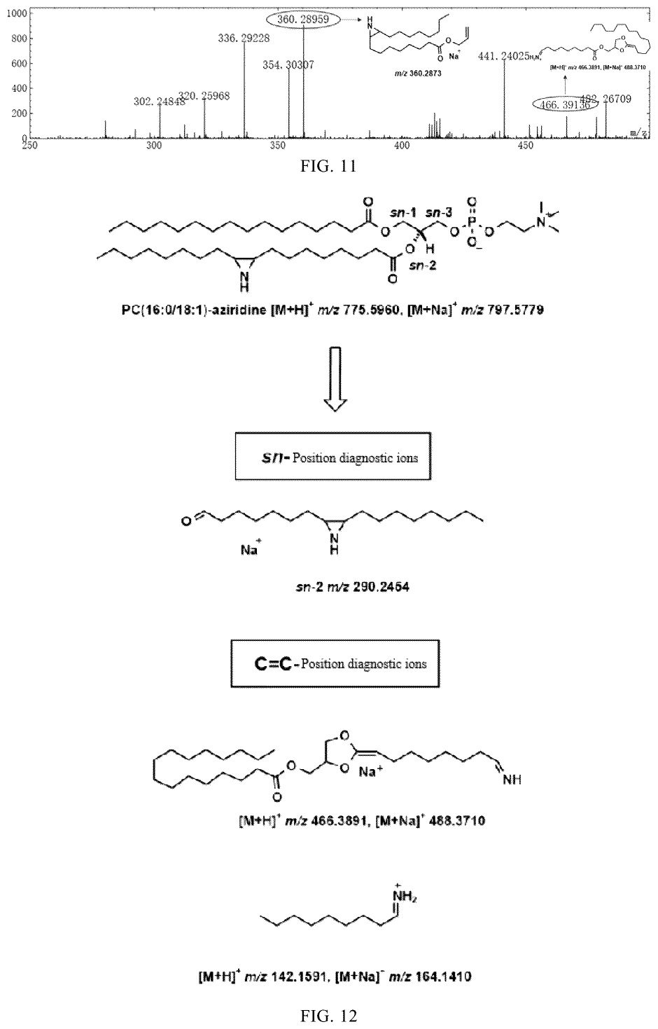

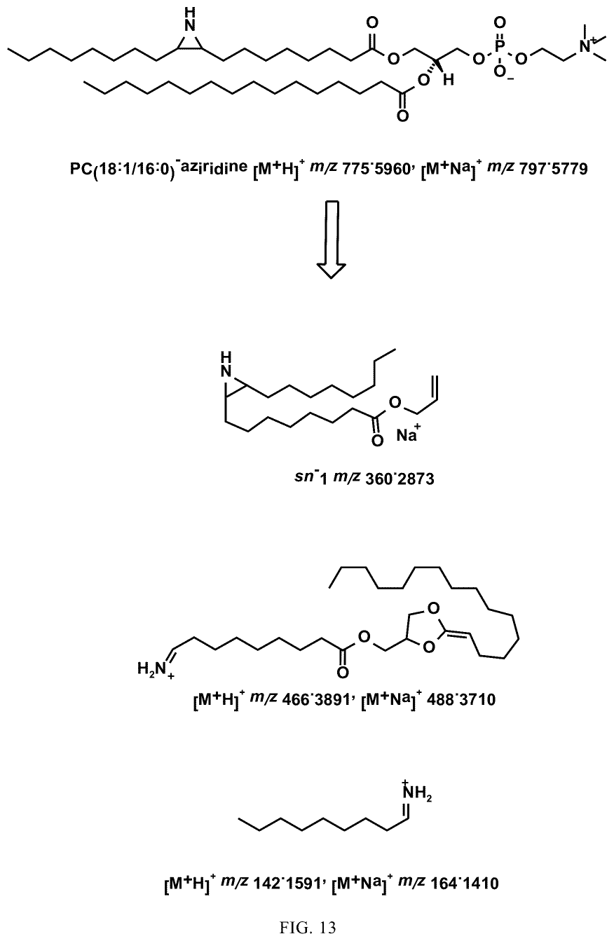

Specifically, PC16:0/18:1 (9Z) having a carbon-carbon double bond at an sn-2 position could obtain a daughter ion of m/z=290 based on a process shown in FIG. 12. PC18:1 (9Z)/16:0 having a carbon-carbon double bond at an sn-1 position could obtain a daughter ion of m/z=360 based on a process shown in FIG. 13, and the sn-position of the carbon-carbon double bond could be determined by the above two characteristic peaks.

### c.3. Identification of Cis-trans Isomer Orientation of Carbon-Carbon Double Bond

The cis-trans isomer orientation of the carbon-carbon double bond could be identified using the ion mobility spectrometer 2. In particular, since the aziridine ring was rigid, the aziridination reaction could increase a structural difference between molecules having different cis-trans configurations, thereby increasing a difference in ion mobility rates of different molecules and separating the migration spectrum peaks.

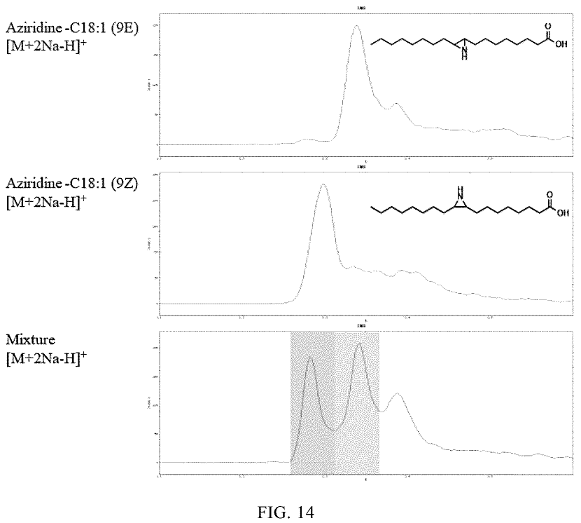

FIG. 14 is a UMA ion mobility spectrometry spectrum (from top to bottom) obtained from aziridinated C18:1 (9E), C18:1 (9Z), and a mixture of C18:1 (9E) and C18:1 (9Z). As shown in FIG. 14, peak positions of molecules having different cis-trans configurations in the ion mobility spectrum were obviously staggered, and this identification method had a good resolution for molecules having different cis-trans configurations.

### d) Over-derivatization

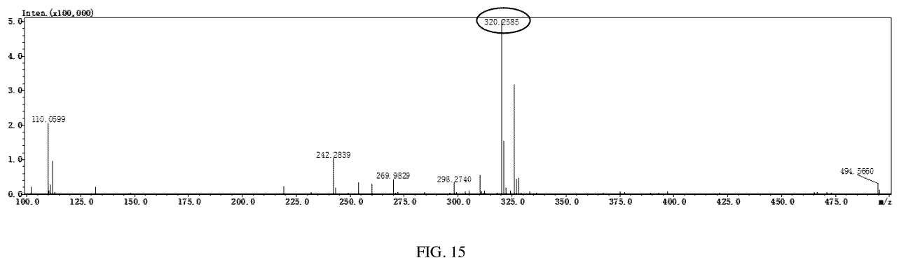

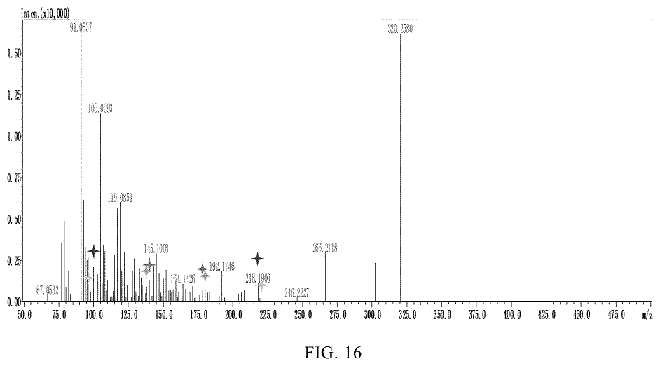

FIGS. 15 to 16 are mass spectra of aziridinized C20:4 (5Z, 8Z, 11Z, 14Z). Referring to FIG. 15, the aza-Prilezhaev reaction could effectively prevent the carbon-carbon double bond from being subjected to over-derivatization, so that only a single carbon-carbon double bond in each molecule of the unsaturated organic compound was reacted in many cases, making the spectrum simpler and easier to analyze. Referring to FIG. 16, eight characteristic mass peaks corresponding to four carbon-carbon double bonds in the mass spectrum were still clearly distinguishable.

According to the above method, the method for identifying an unsaturated organic compound according to the present embodiment has at least one of the following advantages.

a. Many types of identifiable information: a position of a carbon-carbon double bond in a fatty acyl chain, a sn-position of the fatty acyl chain, and a cis-trans isomer orientation of the carbon-carbon double bond can be determined.
b. Good substrate versatility: applicable to various substrates such as FA, GP, ST, SP, and GL.
c. Mild reaction condition: metal catalysts and additional additives are not required, UV, electrolysis, high temperature, inert atmosphere, and dry environment are not required, and there is no strong redox agent. The reaction can be completed quickly at room temperature or slightly increased temperature, and a reagent and a product have good stability.
d. High conversion rate: the conversion rate can reach more than 90%.
e. Few side reactions and enhanced ionization efficiency.
f. Minimal over-derivatization: usually only a single carbon-carbon double bond in each fatty acyl of an unsaturated organic compound is reacted, or by optimizing reaction conditions (such as temperature and time), only a single carbon-carbon double bond in each fatty acyl is reacted. A spectrum is simple and easy to analyze.

### Second Embodiment

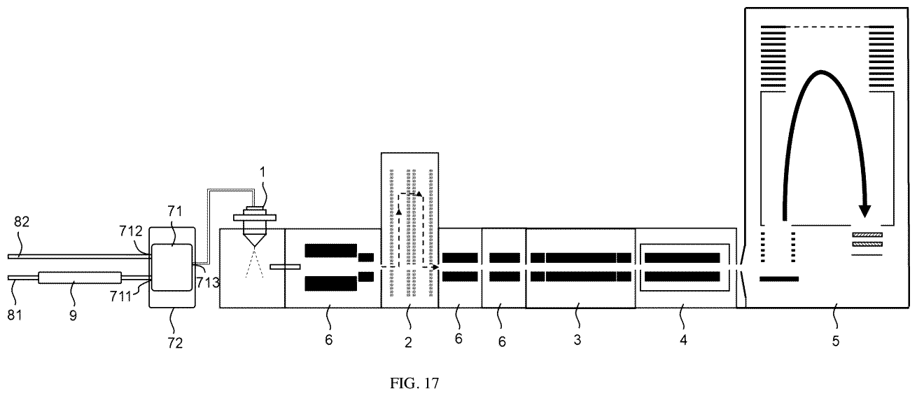

FIG. 17 is a system diagram of a mass spectrometry system for on-line analysis of a derivatization product according to a second embodiment of the present disclosure. A difference from the first embodiment is that the present embodiment uses a derivatization reactor 7 running on-line to implement the aza-Prilezhaev reaction.

Specifically, referring to FIG. 17, the derivatization reactor 7 includes a communication device 71 and an acceleration control unit 72. The communication device 71 has a first liquid inlet 711, a second liquid inlet 712, and a product outlet 713. The first liquid inlet 711 communicates with a liquid inlet pipeline for sample 81 for injection of a sample. The second liquid inlet 712 communicates with a liquid inlet pipeline for derivatization reagent 82 for injection of the derivatization reagent. The product outlet 713 is connected to the ion source 1 to convey the derivatization product generated by the derivatization reaction to the ion source 1.

The acceleration control unit 72 can be any suitable type of a device for increasing molecular collisions, such as a heating, microwave, ultrasound, oscillation, or laser device. Regarding the heating, any suitable type of a temperature control unit, such as a microwave heating device, a water bath, and an oil bath can be used to control the reaction temperature of the derivatization reaction, and especially the reaction temperature is controlled within a temperature range of 20° C. to 100° C. to accelerate the reaction and reduce the occurrence of side reactions.

In the present embodiment, the liquid chromatography device can be combined with an IMS-MS/MS mass spectrometry system to form LC-IMS-MS/MS. Specifically, the liquid chromatography device communicates with the first liquid inlet 711, and a chromatography column 9 of the liquid chromatography device is disposed in the liquid inlet pipeline for sample 81. By combining to form LC-IMS-MS/MS, it is more convenient and quick to complete rapid and high-sensitivity analysis of various lipids through once injection.

The above embodiments are merely exemplary embodiments of the present disclosure and are not intended to limit the present disclosure. Any modifications, equivalent substitutions and improvements made within the spirit and principles of the present disclosure shall be included in the protection scope of the present disclosure.

1. A method for identifying an unsaturated organic compound, the method comprising:
a derivatization reaction step of aziridinating a carbon-carbon double bond in the unsaturated organic compound by an aza-Prilezhaev reaction to obtain a derivatization product, wherein a derivatization reagent is used in the aza-Prilezhaev reaction containing a compound represented by the following general formula (1): 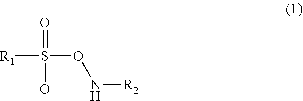
in General Formula (1),
R₁ is selected from a substituted or unsubstituted aromatic group having 6 to 18 ring-forming carbon atoms, and
R₂ is selected from H, a substituted or unsubstituted linear alkyl, alkoxy, or thioalkyl group having 1 to 20 carbon atoms, a substituted or unsubstituted branched or cyclic alkyl, alkoxy, or thioalkyl group having 3 to 20 carbon atoms, a substituted or unsubstituted alkenyl or alkynyl group having 2 to 20 carbon atoms, or a substituted or unsubstituted aromatic or heteroaromatic ring system having 5 to 30 aromatic ring atoms;
a dissociation step of dissociating the ionized derivatization product such that the ionized derivatization product is cleaved at a site corresponding to an original site having the carbon-carbon double bond to obtain a plurality of daughter ions; and
a mass analysis step of measuring mass numbers of the plurality of daughter ions to determine a position of the carbon-carbon double bond in the unsaturated organic compound.

2. The method for identifying an unsaturated organic compound according to claim 1, wherein R₂ has a mass number greater than 80 Da.

3. The method for identifying an unsaturated organic compound according to claim 2, wherein the compound represented by the general formula (1) is as follows:

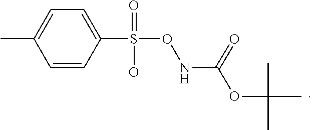

4. The method for identifying an unsaturated organic compound according to claim 1, wherein a solvent of the derivatization reagent is an acidic solvent.

5. The method for identifying an unsaturated organic compound according to claim 4, wherein the acidic solvent is one of trifluoroethanol, hexafluoroisopropanol, and perfluoro-tert-butanol, or a combination thereof.

6. The method for identifying an unsaturated organic compound according to claim 1, wherein a reaction temperature of the aza-Prilezhaev reaction is 20° C. to 100° C.

7. The method for identifying an unsaturated organic compound according to claim 1, wherein the unsaturated organic compound is an unsaturated lipid.

8. The method for identifying an unsaturated organic compound according to claim 7, wherein the unsaturated lipid is fatty acyl, glycerolipid, glycerophospholipid, sphingolipid, a sterol lipid, a prenol lipid, a saccharolipid, or polyketide.

9. A mass spectrometry system, comprising:
a derivatization reactor configured to mix and react a sample with an aza-prilezhaev derivatization reagent, and a carbon-carbon double bond in an unsaturated organic compound in the sample is aziridinated by an aza-Prilezhaev reaction to obtain a derivatization product, the derivatization reagent containing a compound represented by the following general formula (1): 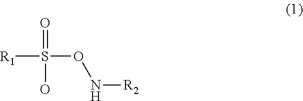
in the general formula (1),
R₁ is selected from a substituted or unsubstituted aromatic group having 6 to 18 ring-forming carbon atoms, and
R₂ is selected from H, a substituted or unsubstituted linear alkyl, alkoxy, or thioalkyl group having 1 to 20 carbon atoms, a substituted or unsubstituted branched or cyclic alkyl, alkoxy, or thioalkyl group having 3 to 20 carbon atoms, a substituted or unsubstituted alkenyl or alkynyl group having 2 to 20 carbon atoms, or a substituted or unsubstituted aromatic or heteroaromatic ring system having 5 to 30 aromatic ring atoms;
an ion source configured to receive and ionize the derivatization product;
a dissociation device configured to dissociate the derivatization product ionized by the ion source such that the derivatization product is cleaved at a site corresponding to an original site having the carbon-carbon double bond to obtain a plurality of daughter ions; and
a mass spectrometer configured to measure mass numbers of the plurality of daughter ions to determine a position of the carbon-carbon double bond in the unsaturated organic compound.

10. The mass spectrometry system according to claim 9, wherein R₂ has a mass number greater than 80 Da.

11. The mass spectrometry system according to claim 10, wherein the compound represented by the general formula (1) is as follows:

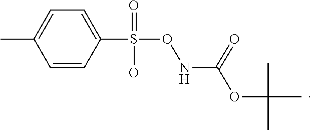

12. The mass spectrometry system according to claim 9, wherein the ion source is an electrospray ionization source, a nanoelectrospray ionization source, a desorption electrospray ionization source, an atmospheric pressure chemical ionization source, an atmospheric pressure photoionization source, or a matrix-assisted laser desorption ionization source.

13. The mass spectrometry system according to claim 9, wherein the dissociation device is one or more of a high-energy collision dissociation device, a collision induced dissociation device, an oxygen attachment dissociation device, a hydrogen attachment dissociation device, an electron capture dissociation device, a radical directed dissociation device, an ultraviolet photodissociation device, and a charge-remote fragmentation device.

14. The mass spectrometry system according to claim 13, wherein the dissociation device is the collision induced dissociation device, and dissociation energy of the collision induced dissociation device is 30 eV to 40 eV.

15. The mass spectrometry system according to claim 9, wherein the derivatization reactor is an off-line reaction device and includes
a reaction vessel in which the sample and the derivatization reagent are mixed, and
an acceleration control unit configured to accelerate collision of molecules in the reaction vessel.

16. The mass spectrometry system according to claim 9, wherein the derivatization reactor is an on-line reaction device and includes
a communication device,
a first liquid inlet disposed in the communication device and communicating with a liquid inlet pipeline for the sample,
a second liquid inlet disposed in the communication device and communicating with a liquid inlet pipeline for the derivatization reagent,
an acceleration control unit configured to accelerate collision of molecules in the communication device, and
a product outlet disposed in the communication device and through which the derivatization product is to be conveyed to the ion source.

17. The mass spectrometry system according to claim 16, wherein the acceleration control unit is one or more of a temperature control unit, an ultrasonic device, a microwave device, an infrared device, and an oscillation device.

18. The mass spectrometry system according to claim 17, wherein the temperature control unit controls a reaction temperature of the aza-Prilezhaev reaction at 20° C. to 100° C.

19. The mass spectrometry system according to claim 18, further comprising:
a liquid chromatography device disposed in the liquid inlet pipeline for the sample.

20. The mass spectrometry system according to claim 9, further comprising:
a mass filter disposed between the ion source and the dissociation device.

21. The mass spectrometry system according to claim 9, further comprising:
an ion mobility spectrometer disposed between the ion source and the dissociation device.
